(19) United States
(12) Patent Application Publication
(10) Pub. No.: US 2023/0314358 A1
(43) Pub. Date: Oct. 5, 2023

(54) INSTRUMENT AND METHOD FOR ANALYSING A COMPLEX MEDIUM IN ORDER TO DETERMINE ITS PHYSICOCHEMICAL PROPERTIES

(71) Applicants: UNIVERSITE GRENOBLE ALPES, Saint-Martin-d'Hères (FR); CENTRE NATIONAL DE LA RECHERCHE SCIENTIFIQUE, Paris (FR); INSTITUT POLYTECHNIQUE DE GRENOBLE, Grenoble (FR); UNIVERSITE SAVOIE MONT BLANC, Chambery (FR)

(72) Inventors: Pascal Xavier, Corenc (FR); Dominique Rauly, Grenoble (FR); Eric Chamberod, Poisat (FR)

(73) Assignees: UNIVERSITE GRENOBLE ALPES, Saint-Martin-d'Hères (FR); CENTRE NATIONAL DE LA RECHERCHE SCIENTIFIQUE, Paris (FR); INSTITUT POLYTECHNIQUE DE GRENOBLE, Grenoble (FR); UNIVERSITE SAVOIE MONT BLANC, Chambery (FR)

(21) Appl. No.: 18/016,977

(22) PCT Filed: Jul. 20, 2021
(86) PCT No.: PCT/FR2021/051356

§ 371 Date: Jan. 19, 2023

(30) Foreign Application Priority Data
Jul. 20, 2020 (FR) FR2007582

(51) Int. Cl. G01N 27/22

(52) U.S. Cl. CPC G01N 27/223; G01N 27/221

(57) ABSTRACT

Methods, apparatuses and systems for radio frequency identification (RFID)-enabled information collection are disclosed, including an enclosure, a collector coupled to the enclosure, an interrogator, a processor, and one or more RFID field sensors, each having an individual identification, disposed within the enclosure. In operation, the interrogator transmits an incident signal to the collector, causing the collector to generate an electromagnetic field within the enclosure. The electromagnetic field is affected by one or more influences. RFID sensors respond to the electromagnetic field by transmitting reflected signals containing the individual identifications of the responding RFID sensors to the interrogator. The interrogator receives the reflected signals, measures one or more returned signal strength indications (“RSSI”) of the reflected signals and sends the RSSI measurements and identification of the responding RFID sensors to the processor to determine one or more facts about the influences. Other embodiments are also described.

The present invention generally relates to a probe-type instrument and to a method for analyzing depthwise a complex medium to determine the physico-chemical properties thereof.

By complex medium, what is meant, in the context of the present invention, is media such as soils, especially agricultural soils, footwalls, or snowpacks. These are essentially natural media that may be stratified.

To analyze such media depthwise, it is known in the art to use methods allowing the presence of obstacles or the boundary between two distinct media to be located without analyzing the physico-chemical properties of these media, as taught in United States pat. US 8,933,789.

Well-known methods, such as the measurement of the dielectric properties of the medium to be analyzed as a function of the frequency of the sine-wave generator, are also known in the art. Measurement of the dielectric properties of the medium to be analyzed as a function of frequency allows the nature of the materials present in the location where the measurement is taken to be accurately detected. The method of time domain reflectometry (TDR) is also known in the art. This method consists of sending a voltage pulse into the medium to be analyzed and recording the reflected voltage. It allows the variation in the electrical permittivity of the medium as a function of depth (changes of media, layers) to be detected. However, these conventional methods have the drawback of not allowing complete analysis of a complex medium depthwise: they allow only a single parameter to be analyzed, electrical capacitance in the case of the first, in the location where the measurement is taken, while with TDR, although a depthwise analysis is possible, it is only able to measure the depthwise variation in electrical permittivity when the conductivity of the medium is not too high.

In order to be able to obtain a depthwise image of complex media via a single invasive measurement using a single instrument that detects the variation in a number of physical parameters, the Applicant has developed a profile-determining instrument and method based on measurement, at various frequencies, of the distribution, along the axis of propagation, of the amplitude of a standing electric field wave created in a transmission line inserted into the stratified medium to be studied. The propagation speed of the wave and its attenuation are determined for different layers of the medium to be analyzed. This speed and this attenuation depend directly on the dielectric constant εᵣ and on the electrical conductivity σ of the medium. Analysis of the frequency-domain, and optionally time-domain, variation in these two parameters and measurement in parallel of temperature profile makes it possible to deduce, for each layer, secondary physical parameters that depend on the targeted application: for example moisture content or liquid water content, salinity, input concentration, organic matter content, or water equivalent (for snow).

More particularly, one subject of the present invention is an instrument for analyzing a complex medium, in particular a natural medium, which may be stratified, in order to determine its physico-chemical properties, this instrument comprising:
a (so-called two-conductor) transmission line of length L comprising two parallel metal conductors that are placed in the medium to be analyzed and arranged facing each other symmetrically with respect to a central axis x, and being terminated by an open circuit allowing total reflection of the wave,
a radio-frequency (radio-frequency usually being abbreviated RF) sine-wave generator delivering a frequency f varying between 2 MHz and 2 GHz, for supplying the metal conductors,
a plurality N of RF detectors arranged between the metal conductors and regularly spaced apart from each other, each RF detector being provided with a printed antenna, said RF detectors and their associated printed antennas being regularly distributed along the transmission line and spaced apart from each other, each RF detector being capable of converting the power of the signal captured by the antenna with which it is associated into a DC voltage, whatever the frequency delivered by the generator,
a supervisor board for controlling the RF sine-wave generator and the plurality of RF detectors and their associated printed antennas.

The spatial measurement pitch is discrete: in other words, the RF detectors and their associated antennas are regularly distributed along the transmission line and spaced apart from each other.

Advantageously, the instrument according to the invention may also comprise at least one microcontroller comprising an analog-to-digital converter, said analog-to-digital converter being intended to convert each voltage measured by the RF detectors into a digital value intended to be transmitted to the supervisor board.

Advantageously, the printed antennas may be arranged between said metal conductors off-center with respect to the central axis x of the transmission line. This off-center location of the printed antennas increases the signal (captured by an antenna)/noise ratio.

Advantageously, the RF detectors may be grouped together in modules each comprising a string of 8 series-connected RF detectors and one microcontroller.

Another subject of the present invention is a method for analyzing, as a function of depth x, a complex medium comprising at least one layer of solid and/or liquid material, to determine the physical properties of said layer of solid and/or liquid material. If the complex medium comprises a plurality of layers, each layer is considered to be a continuous medium (therefore there are no abrupt discontinuities in its physical properties within the layer). The method according to the invention comprises the following steps:
inserting into the complex medium to be analyzed, an instrument according to the invention by placing it in said complex medium, the surface of said complex medium defining the origin x=0 of the analysis;
supplying, using said sine-wave generator, the transmission line with a sinusoidal signal of frequency f varying between 2 MHz and 2 GHz, so as to generate an electric field E, inducing a current in each of the printed antennas the power of which is converted into a DC voltage by the RF detector associated therewith, propagation of said electric field E along the transmission line and between the metal conductors of said instrument resulting in appearance of at least one standing wave of wavelength λ and amplitude V₂₀(z) dependent on the abscissa z, with z=L-x;
converting, by means of the analog-to-digital converter (ADC), the various DC voltages thus obtained by the RF detectors into digital values;
transmitting the digital values thus obtained to the supervisor board, which is programmed to carry out post-processing thereon and to convert them into a curve representing the variation in the amplitude V₂₀(z) of the standing wave in the layer of solid and/or liquid material, along the abscissa z, with z=L-x (in other words, the N RF detectors indirectly return a spatially sampled measurement of V₂₀(z));
determining, by interpolation of the values, the depths x at which minimum voltages and maximum voltages appear and the amplitude of the maximum values of V₂₀(z);
computing the complex electrical permittivity ε* = εᵣ - j.σ/(2.π.f.ε₀) of said layer of solid and/or liquid material as a function of the depth x and for each measurement frequency f; with
ε₀ designating the dielectric constant of vacuum,
εᵣ (real part of the complex permittivity ε*) designating the dielectric constant in said layer of solid and/or liquid material, and
σ designating the electrical conductivity σ of said layer of solid and/or liquid material,
f designating the frequency of the sinusoidal signal, and
j designating the mathematical operator such that j²=-1,
this step of computing complex electrical permittivity ε* comprising two steps: - a first step comprising determining the half-wavelength λ/2 of the standing wave, corresponding to the distance between two successive minima of the curve of variation in the amplitude V₂₀(z) of the standing wave, and computing the speed c of the standing wave and the dielectric constant εᵣ in said layer of solid and/or liquid material; and
a second step for computing the exponential attenuation α of the standing wave between two successive maxima of the curve of variation in the amplitude V₂₀(z) of the standing wave, this attenuation directly depending on the imaginary part of the complex electrical permittivity ε* of said layer of solid and/or liquid material, said imaginary part itself depending directly on the electrical conductivity σ of said layer of solid and/or liquid material.

Superposition of the forward-propagating wave and back-propagating wave results in the appearance of a standing wave (its amplitude at a given abscissa z remains constant, but of course the standing wave remains sinusoidal over time).

For a transmission line with losses, the origin z=0 of the axis being at the load, it may be shown that the complex general expression for this steady-state voltage may be written as in equation (1):

$$\text{V} \left(\text{z}\right) = \text{Vi} \text{.} \left({\text{e}}^{\text{γ} \text{z}} + {\text{Γ}}_{\text{L} \text{.}} {\text{e-}}^{\text{γ} \text{z}}\right) \qquad \text{(1)}$$

with
Vi defining the voltage delivered by the sinusoidal voltage generator to the input of the transmission line, the input of the transmission line being used as reference point to define the phases,
Γ_{L} being the coefficient of reflection from the load, which is substantially equal to 1 because the load is an open circuit,
z defining the abscissa according to the relationship z=L-x.

The general form of the amplitude of V(z) is then as shown in FIG. 3, which illustrates a curve obtained by discretization over N measurement points of the following developed version of equation (1): [symbol] with Vi, α (attenuation in Np/m), Γ_{L} (of the order of 1) and β (phase constant in rad/m) being parameters which depend on the medium. The effective dielectric constant (taking line geometry into account) may be deduced directly from β, and electrical conductivity may be deduced from α via transmission-line formulae. It will be noted that δ expresses the fact that the last sensor is not at the abscissa z=0.

In practice, since the period of the observed wave is equal to λ/2, the effective εᵣ for each detected spatial half-period is deduced directly and, subsequently, the dielectric constant εᵣ, the relationship between the effective εᵣ and εᵣ having been determined by calibrating the instrument in four media of known dielectric constant (air, dry sand, glass beads and ethyl alcohol). The envelope of the curve further allows α to be deduced and therefore the conductivity σ to be determined. To this end, on the one hand the distances between minima and on the other hand the decrease in the amplitude maxima are measured.

Advantageously, the method according to the invention may further comprise an experimental step of measuring the temperature of said stratified medium to be analyzed, in order to increase the precision of the analysis. This step will possibly for example be carried out using a subsidiary temperature profiler.

The frequency f varying between 2 MHz and 2 GHz is adjustable, as illustrated in the embodiments below.

According to a first embodiment of the method according to the invention, the medium to be analyzed is damp agricultural soil, or soil containing granular waste or a peat bog. In this embodiment, the method according to the invention further comprises a step of computing with analytical equations moisture content based on the dielectric constant εᵣ in the one or more layers of solid and/or liquid material, and monitoring the variation as a function of time in this content by making a request to a database fed with the measurements of the instrument.

Preferably, the method according to this embodiment, which is suitable for analyzing moist soil, may further comprise the following steps:
measuring the profile of the conductivity σ of the medium to be analyzed, this profile being computed based on the imaginary part of the complex permittivity in the one or more layers of solid or liquid material, and monitoring the variation in this conductivity as a function of time and depth x by making a request to a database fed with the measurements of the instrument,
based on the measurements carried out in the preceding steps, deducing, by analyzing the drift as a function of time in moisture content and conductivity σ for each measurement frequency and by comparing it with analytical equations derived from a physico-chemical model of the medium, the content of organic matter in the one or more layers of the medium to be analyzed, and the level of inputs (N, P, K) and the salinity level.

According to a second embodiment of the method according to the invention, the medium to be analyzed is the type of stratified natural medium referred to as a snowpack. In this embodiment, the method according to the invention further comprises the following steps:
measuring the losses and the dielectric constant of the snowpack about a frequency of 2 GHz by determining the maxima and minima of the standing wave at high frequency in said one or more layers of solid and/or liquid material;
determining the amplitude of the electric field detected at a frequency below 2 GHz, in order to deduce therefrom the dielectric constant of the medium in the one or more layers of solid and/or liquid material;
computing, based on the various values determined in the preceding steps, the proportion by volume of ice, water and air in said one or more layers of solid material, snow height and values of the liquid water content (LWC) and snow water equivalent (SWE) in the one or more layers of solid and/or liquid material being deduced directly therefrom.

Yet another subject of the present invention is use of the method according to the invention to determine whether the analyzed medium (in particular if it is a question of a natural medium) is stratified and comprises at least two layers of solid and/or liquid material that are different from each other in nature: whether or not the medium analyzed is stratified is then determined by observing the appearance of a change/break in the variation in V₂₀(z), in the wavelength λ and in the attenuation α of the standing wave, these varying depending on the nature of the layers of solid and/or liquid material of the analyzed medium. Given a sufficient number of sensors (the number of which depends on the precision desired in respect of dielectric constant), it is possible to determine whether the line has been placed in a stratified medium because at least two different wavelengths will be detected.

Other advantages and particularities of the present invention will result from the following description, which is given by way of non-limiting example with reference to the appended figures and the examples:

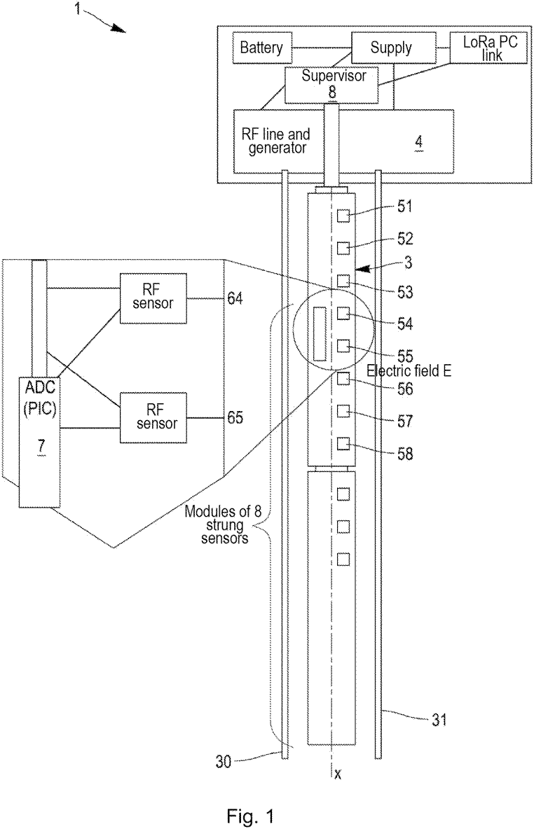

FIG. 1is a schematic cross-sectional representation of an example of a probe-type instrument according to the invention;

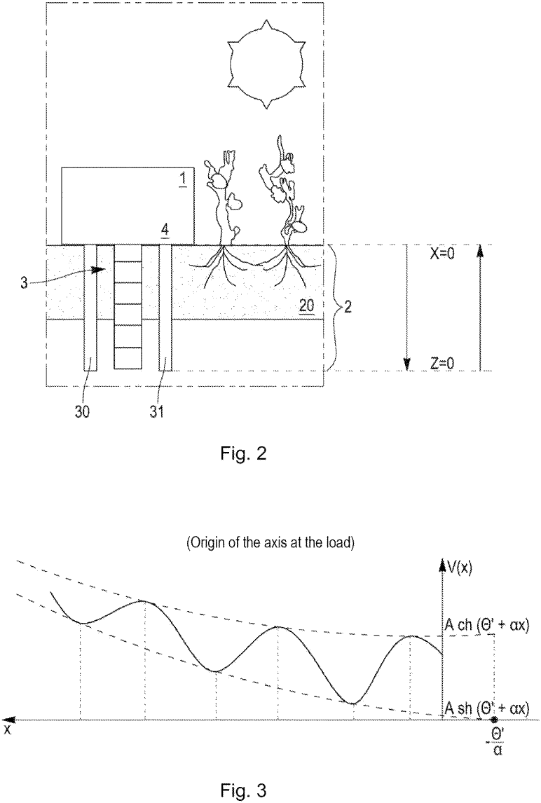

FIG. 2is a schematic cross-sectional representation of the probe-type instrument illustrated in FIG. 1 placed in a complex moist soil;

FIG. 3is a representation of the typical spatial variation in the amplitude of the standing voltage wave V₂₀(z) in the line in the presence of a non-zero conductivity;

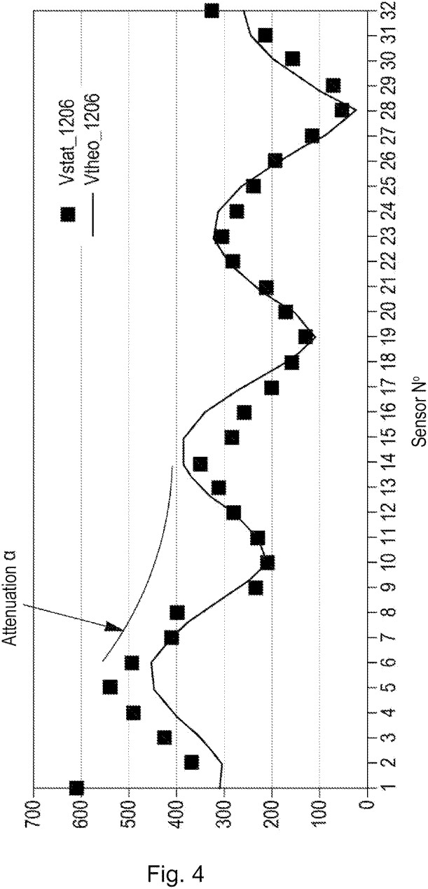

FIG. 4is an example of measurements made at 1206 MHz showing the variation in the amplitude V₂₀(z) of the standing wave in a tract of agricultural land (the small squares are experimental measurements and the continuous line is a model curve) as a function of depth x;

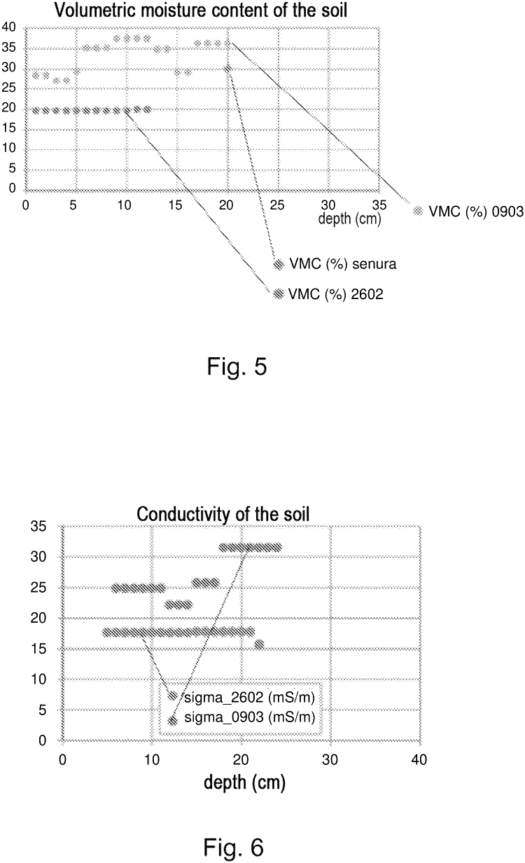

FIG. 5shows the variation in the moisture content determined, in accordance with the method according to the invention, based on two series of measurements using the instrument according to the invention;

FIG. 6shows the variation in conductivity obtained, in accordance with the method according to the invention, based on the same series of measurements as in FIG. 5;

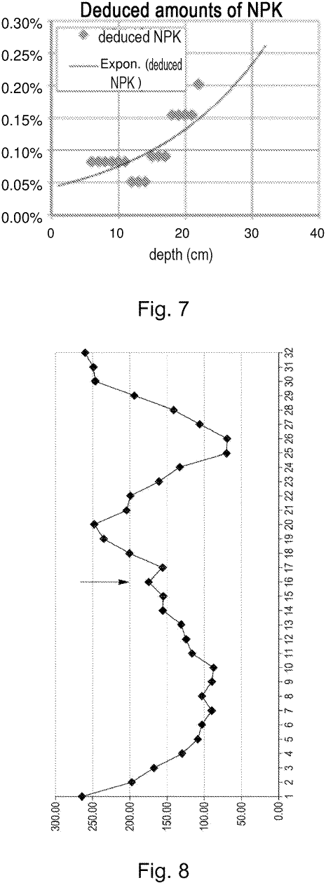

FIG. 7shows the variation in the level of inputs (N, P, K) in the soil, as deduced from the results presented in FIGS. 5 and 6;

FIG. 8is an example of measurements taken at 585 MHz, showing the variation in the amplitude V₂₀(z) of the standing wave in air then in dry spoil, in accordance with the arrangement illustrated by FIG. 2;

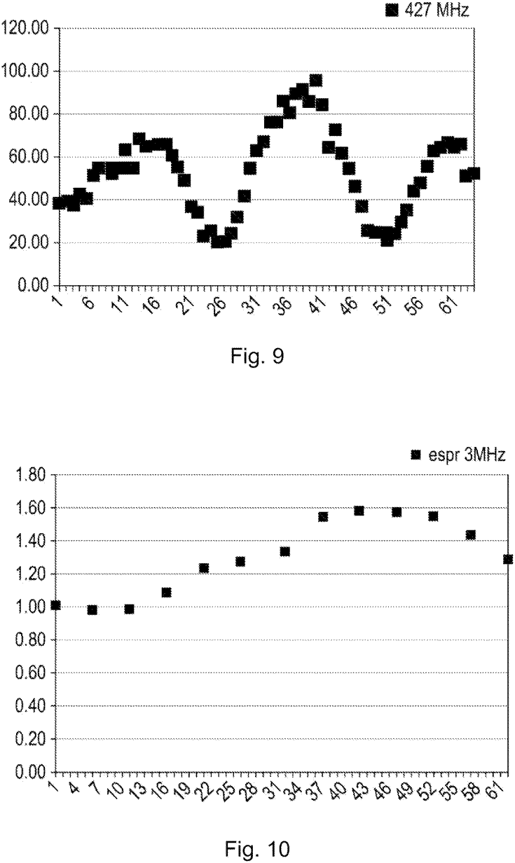

FIG. 9 shows the variation in the amplitude of the standing wave in microvolts as a function of depth x, at a frequency f of 427 MHz, for a snowpack (example 3),

FIG. 10shows the variation in the dielectric constant at each abscissa, at f=3 MHz, for the snowpack of example 3;

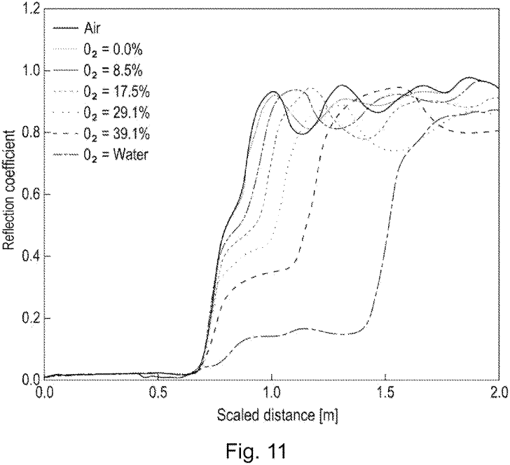

FIG. 11 shows the variation in the amplitude of the voltage reflected to ground level from dry and moist sand, as measured using a TDR probe (example 4).

Figures FIG. 1 and FIG. 2show one example of a probe-type measuring instrument according to the present invention. FIG. 3 was discussed in the above part of the description. Figures FIG. 4 to FIG. 11 are discussed in examples 1 to 3 and the comparative example below.

This instrument 1 comprises:
a two-conductor transmission line 3 comprising two parallel metal conductors 30, 31 that are arranged facing each other symmetrically with respect to a central axis x, and being terminated by an open circuit allowing total reflection of the wave,
an RF sine-wave generator 4 for supplying the metal conductors 30, 31,
a plurality of N RF detectors 51, 52, 53, 54, 55, 56, 57, 58 arranged between the metal conductors 30, 31 and regularly spaced apart from each other,
each RF detector 51, 52, 53, 54, 55, 56, 57, 58 being provided with a printed antenna 61, 62, 63, 64, 65, 66, 67, 68, these printed antennas 61, 62, 63, 64, 65, 66, 67, 68 preferably being arranged between the metal conductors 30, 31 off-center with respect to the central axis x of the transmission line 3,
each RF detector 51, 52, 53, 54, 55, 56, 57, 58 being capable of converting the power of the signal captured by the antenna 61, 62, 63, 64, 65, 66, 67, 68 with which it is associated into a DC voltage;
in the exemplary embodiment shown in figures FIG. 1and FIG. 2, the RF detectors 51, 52, 53, 54, 55, 56, 57, 58 are grouped together in modules each comprising a string of 8 RF detectors 51, 52, 53, 54, 55, 56, 57, 58 and one microcontroller 7 ADC/PIC.

This microcontroller 7 comprises an analog-to-digital converter for converting each voltage measured by the RF detectors 51, 52, 53, 54, 55, 56, 57, 58 into a digital value intended to be transmitted to a supervisor board 8, which controls the RF sine-wave generator 4 and the plurality of RF detectors 51, 52, 53, 54, 55, 56, 57, 58 and their associated printed antennas 61, 62, 63, 64, 65, 66, 67, 68.

### EXAMPLES

### Example 1: Determining Using the Method According to the Invention the Variation in the Amplitude V₂₀(z) of the Standing Wave in a Tract of Moist Sandy Agricultural Soil

It is sought to determine, using the method according to the invention, the depth profile of a tract of moist sandy soil.

Analyses carried out in 2018 by SENuRA (acronym of Station d′Expérimentation Nucicole Rhône-Alpes, a center of nut-farming research) using a commercial ENVIRON-SCAN capacitive probe from the company SENTEK showed that it was a question of sandy-loamy-clayey soil of 72% by weight sand, 20% by weight silt and 8% by weight clay, this soil being quite stony.

The organic matter content MO was 22 g/kg, and the N, P and K input contents were read as follows:
N = 1.2 g/kg,
P = 0.95 g/kg, and
K = 0.322 g/kg.

The instrument according to the invention such as shown in [FIG. 1] and [FIG. 2] was placed in the tract of land by digging pilot holes with an auger and, if necessary, with a perforator, then inserting the instrument vertically into the holes thus formed.

[FIG. 4] shows very visibly an exponential attenuation of the amplitude of the standing wave due to the conductivity σ of the medium and a gradual decrease with depth in the guided wavelength due to an increase in moisture content with x, this being consistent with what was expected and measured by a capacitive probe.

The measurement points have been represented by squares, and the model resulting from the equation for V₂₀(z) and from taking into account the moisture-content gradient in the variation in the dielectric constant has been represented by a solid line.

The measurement points have been represented by squares, and the model resulting from the equation for V₂₀(z) and from taking into account the moisture-content gradient in the variation in the dielectric constant has been represented by a solid line.

Two series of similar measurements were carried out on Feb. 26, 2021 (date on which the instrument according to the invention was sunk into the ground to a height H of 22 cm) and Mar. 09, 2021 (date on which the instrument according to the invention was sunk into the ground at a height H of 24 cm because of the presence of pebbles). Between these two dates, manure was spread once and it rained a little.

The moisture contents designated in FIG. 5 “VMC (%) 2602” and “VMC (%) 0903” (VMC standing for Volumetric Moisture Content) were calculated analytically based on the values of the dielectric constant εᵣ obtained from these two series of measurements, respectively. In parallel, SENuRA measured moisture content using the aforementioned “Environ-Scan” capacitive probe from SENTEK (single diamond-shaped point designated “VMC(%) senura” in FIG. 5).

FIG. 5 shows that the moisture contents calculated analytically in accordance with the method according to the invention are of the same order of magnitude as the moisture content measured by SENuRA with the capacitive probe (of the order of 30% for soil saturated with water), given that experimental uncertainty is ± 5 to 10%.

Next, for these two series of measurements, the profile of the conductivity σ of the soil was determined from the imaginary part of the complex electrical permittivity ε* and the variation in this conductivity σ as a function of time and depth x was plotted, as illustrated in FIG. 6. Based on the results presented in FIGS. 5 and 6, the level of inputs (N, P, K) into the soil were deduced by analyzing the drift as a function of time in moisture content and conductivity σ for each measurement frequency and by comparing it with analytical equations derived from a physico-chemical model of the medium (see FIG. 7). The level of inputs is in practice computed empirically based on standard measurements using a linear law, in accordance with the teaching of the scientific publication by Vidya D. Ahire et al. “Effect of Chemical Fertilizers on Dielectric Properties of Soils at Microwave Frequency”, in International Journal of Scientific and Research Publications, Volume 5, Issue 5, May 2015, ISSN 2250 -3153.

Between February 26 and March 9, an increase in the conductivity of the soil was observed that may be ascribed to an added percentage of NPK (mass of inputs for an equivalent volume of 1 liter of water). Over the first 22 cm from ground level, the measured amounts were comprised between 0.5 and 2 g/kg, comparable to the values measured for the various inputs N, P and K by SENuRA in June 2018 (N = 1.2 g/kg, P = 0.95 g/kg, and K = 0.322 g/kg). It may moreover be seen, from the exponential shape of the fitted curve passing through the points of FIG. 7, that the fertilizer seemed to have diffused into the soil.

### Example 2: Determining Using the Method According to the Invention the Variation in the Amplitude V₂₀(z) of the Wave When Half the Instrument is Placed in Spoil Saturated With Water, the Other Half Remaining in Air

The aim was to determine the depthwise profile of spoil saturated with water. To this end, the instrument according to the invention such as shown in [FIG. 1] was placed in the ground as shown by [FIG. 2] and the procedure described in example 1 carried out. In accordance with the method according to the invention, the variation in the amplitude of the standing wave in microvolts as a function of depth x was then determined at a frequency f of 585 MHz, the results being illustrated in [FIG. 8].

[FIG. 8] allows direct observation, from the extrema, of an effective dielectric constant of 1.6 for the upper part of the instrument and of 5 for the part inserted into the spoil. This corresponds perfectly to air in the upper part and to a volumetric moisture content of 25% in the part submerged which was water-saturated, which value is almost equal to that measured by gravimetric analysis of the same tract (23%). The interface at x=16 cm between air and spoil may also be seen (indicated by the arrow).

### Example 3: Determining Using the Method According to the Invention the Variation in the Amplitude V₂₀(z) of the Wave When the Instrument is Placed in a Snowpack

The aim was to determine the depthwise profile of a snowpack. To this end, the instrument according to the invention such as shown in [FIG. 1] was placed in the ground as shown by [FIG. 2] and the procedure described in example 1 carried out.

In accordance with the method according to the invention, the variation in the amplitude of the standing wave in microvolts as a function of depth x was then determined at a frequency f of 427 MHz, the results being illustrated in [FIG. 9].

At 3 MHz (see [FIG. 10]) the RF detectors detect an amplitude directly proportional to electric field, which depends on the dielectric constant at each abscissa. The curve of [FIG. 10] represents this dielectric constant, the signal being integrated over 5 detectors in order to decrease the influence of measurement noise.

[FIG. 9] and [FIG. 10] thus allow the complex permittivity at these two frequencies to be computed directly for layers with a thickness larger than or equal to 10 cm. For one of these layers, it was found that εᵣ = 1.52 at 427 MHz and εᵣ = 1.6 at 3 MHz. This was deduced using a mixing law model of the volumetric proportions of ice I = 29.2% and water W = 0.5%, and a density D of 272 kg/m3.

These results are to be compared with those obtained experimentally using a capacitive probe associated with a weighing method: I = 32.4%, W = 0.6%, density D of 330 kg/m³.

### Example 4: (Comparative Example): Determining Moisture Profile in Dry and Wet Sand Using the Prior Art Method of Time Domain Reflectometry (TDR)

The aim was to determine the depthwise profile of sandy soil. To do this, the TDR instrument (typically a probe consisting of a three-pronged metal transmission line supplied by a pulse generator) was placed in the ground. The variation in the amplitude of the voltage reflected to ground level was then determined as illustrated in [FIG. 11].

[FIG. 11] clearly shows that the more the moisture content (as given by the parameter VMC, acronym of Volumetric Moisture Content, here denoted Θ, in %) of the medium increases, the more the reflected pulse is deformed by the losses which the forward-propagating and back-propagating wave is subject to at every point on its path through the three-pronged line, this preventing the profile of moisture content and of conductivity of the measured medium from being determined accurately, especially if the measured medium is stratified.

1. An instrument for analyzing a complex medium to determine the physico-chemical properties thereof, the instrument comprising:
a transmission line having a length L comprising two parallel metal conductors that are placed in the medium to be analyzed and arranged facing each other symmetrically with respect to a central axis x, and being terminated by an open circuit allowing total reflection of a wave,
an RF sine-wave generator delivering a frequency f varying between 2 MHz and 2 GHz, for supplying the metal conductors,
a plurality of RF detectors arranged between the metal conductors and regularly spaced apart from each other, each of the RF detectors being provided with an associated printed antenna, the RF detectors and associated printed antennas being regularly distributed along the transmission line and spaced apart from each other, each of the RF detectors being capable of converting a power of a signal captured by the printed antenna with which the RF detector is associated into a DC voltage,
a supervisor board for controlling the RF sine-wave generator and the RF detectors and associated printed antennas.

2. The instrument as claimed in claim 1, further comprising at least one microcontroller comprising an analog-to-digital converter, the analog-to-digital converter being adapted to convert each voltage measured by the RF detectors into a digital value intended to be transmitted to the supervisor board.

3. The instrument as claimed in claim 1, wherein the printed antennas are arranged between the metal conductors off-center with respect to the central axis of the transmission line.

4. The instrument as claimed in claim 1, wherein the RF detectors are grouped together in modules, each of the modules comprising a string of eight RF detectors and one microcontroller.

5. A method for analyzing, as a function of depth, a complex medium comprising at least one layer of solid and/or liquid material, to determine physical properties of the layer of solid and/or liquid material, the method comprising:
inserting into the complex medium to be analyzed, the instrument as defined in claim 1 by placing the instrument in the complex medium, the surface of the complex medium defining an origin x=0 of analysis;
supplying, using the sine-wave generator, the transmission line with a sinusoidal signal of frequency varying between 2 MHz and 2 GHz, so as to generate an electric field, inducing a current in each of the printed antennas, the power of which is converted into a DC voltage by the RF detector to which the printed antenna is associated, propagation of the electric field E along the transmission line and between the metal conductors of the instrument resulting in appearance of at least one standing wave of wavelength λ and amplitude V₂₀(z) dependent on the abscissa z, with z=L-x;
converting, by means of the analog-to-digital converter, the various DC voltages obtained by the RF detectors into digital values;
transmitting the digital values obtained to the supervisor board, the supervisor board being programmed to carry out post-processing thereon and to convert the digital values into a curve representing a variation in the amplitude V₂₀(z) of the standing wave in the layer of solid and/or liquid material, along the abscissa z, with z=L-x;
determining, by interpolation of the digital values, depths x at which minimum voltages and maximum voltages appear and an amplitude of the maximum values of V₂₀(z);
computing a complex electrical permittivity ε* = εᵣ - j.σ/(2.π.f.ε₀) of the layer of solid and/or liquid material as a function of the depth x and for each measurement frequency f, with
ε₀ designating a dielectric constant of vacuum,
εᵣ (real part of the complex permittivity ε*) designating a dielectric constant in the layer of solid and/or liquid material, and
σ designating an electrical conductivity of the layer of solid and/or liquid material,
j designating a mathematical operator defined so that j²=-1,
wherein the computing comprises:
determining a half-wavelength λ/2 of the standing wave, corresponding to a distance between two successive minima of the curve of variation in the amplitude V₂₀(z) of the standing wave, and computing a speed c of the standing wave and the dielectric constant εᵣ in the layer of solid and/or liquid material;
computing an exponential attenuation α of the standing wave between two successive maxima of the curve of variation in the amplitude V₂₀(z) of the standing wave, the attenuation depending directly on an imaginary part of the complex electrical permittivity ε* of the layer of solid and/or liquid material, the imaginary part depending directly on the electrical conductivity σ of the layer of solid and/or liquid material.

6. The method as claimed in claim 5, further comprising measuring a temperature of the complex medium in a form of stratified medium to be analyzed.

7. The method as claimed in claim 6, wherein the complex medium to be analyzed is a moist soil, the method further comprising:
computing with analytical equations moisture content based on the dielectric constant εᵣ in the at least one layer of solid and/or liquid material, and monitoring a variation as a function of time in the moisture content by making a request to a database fed with measurements of the instrument.

8. The method as claimed in claim 7, further comprising:
measuring a profile of the conductivity σ of the medium to be analyzed, the profile being computed based on the imaginary part of the complex electrical permittivity ε* in the at least one layer of solid and/or liquid material,
based on the measurements carried out in the computing of the moisture content and the measuring of the profile of the conductivity, deducing, by analyzing the drift as a function of time of the moisture content and of the conductivity σ for each measurement frequency and by comparing the drift with analytical equations derived from a physico-chemical model of the medium, the content of organic matter in the at least one layer of the medium to be analyzed, a level of inputs N, P, K, and salinity.

9. The method as claimed in claim 6, wherein the medium to be analyzed is a stratified natural medium referred to as a snowpack, the snowpack comprising at least one the layer of solid material, the method further comprising:
measuring losses and a dielectric constant of the snowpack about a frequency of 2 GHz by determining the maxima and minima of the standing wave at high frequency in the one or more layers of solid and/or liquid material;
determining an amplitude of the electric field detected at a frequency below 2 GHz, in order to deduce therefrom the dielectric constant of the medium in at least one the layer of solid and/or liquid material;
computing, based on the various values determined in the measuring of the losses and the dielectric constant and the determining of the amplitude of the electric field, a proportion by volume of ice, water and air in the at least one layer of solid material, snow height and values of liquid water content and snow water equivalent in the at least one layer of solid material being deducible directly therefrom.

10. The method as claimed in claim 6, comprising determining whether the analyzed medium is stratified and comprises at least two layers of solid and/or liquid material that are different from each other in nature, by observing the appearance of a change in the variation in V₂₀(z), in the wavelength λ and in the attenuation of the standing wave, wherein the variation depends on a nature of the layers of solid and/or liquid material of the analyzed medium.

11. The method as claimed in claim 8, comprising, in the measuring of the profile of the conductivity σ of the medium to be analyzed, monitoring a variation in the conductivity σ as a function of time and depth x by making a request to a database fed with measurements of the instrument.

12. The method as claimed in claim 5, wherein the complex medium to be analyzed is a moist soil, the method further comprising:
computing with analytical equations moisture content based on the dielectric constant εᵣ in the at least one layer of solid and/or liquid material, and monitoring a variation as a function of time in the moisture content by making a request to a database fed with measurements of the instrument.

13. The method as claimed in claim 12, further comprising:
measuring a profile of the conductivity σ of the medium to be analyzed, the profile being computed based on the imaginary part of the complex electrical permittivity ε* in the at least one layer of solid and/or liquid material,
based on the measurements carried out in the computing of the moisture content and the measuring of the profile of the conductivity, deducing, by analyzing the drift as a function of time of the moisture content and of the conductivity σ for each measurement frequency and by comparing the drift with analytical equations derived from a physico-chemical model of the medium, the content of organic matter in the at least one layer of the medium to be analyzed, a level of inputs N, P, K, and salinity.

14. The method as claimed in claim 5, wherein the medium to be analyzed is a stratified natural medium referred to as a snowpack, the snowpack comprising at least one layer of solid material, the method further comprising:
measuring losses and a dielectric constant of the snowpack about a frequency of 2 GHz by determining the maxima and minima of the standing wave at high frequency in the one or more layers of solid and/or liquid material;
determining an amplitude of the electric field detected at a frequency below 2 GHz, in order to deduce therefrom the dielectric constant of the medium in at least one layer of solid and/or liquid material;
computing, based on the various values determined in the measuring of the losses and the dielectric constant and the determining of the amplitude of the electric field, a proportion by volume of ice, water and air in the at least one layer of solid material, snow height and values of liquid water content and snow water equivalent in the at least one layer of solid material being deducible directly therefrom.

15. The method as claimed in claim 5, comprising determining whether the analyzed medium is stratified and comprises at least two layers of solid and/or liquid material that are different from each other in nature, by observing the appearance of a change in the variation in V₂₀(z), in the wavelength λ and in the attenuation of the standing wave, wherein the variation depends on a nature of the layers of solid and/or liquid material of the analyzed medium.

16. The method as claimed in claim 13, comprising, in the measuring of the profile of the conductivity σ of the medium to be analyzed, monitoring a variation in the conductivity σ as a function of time and depth x by making a request to a database fed with measurements of the instrument.

17. The instrument as claimed in claim 2, wherein the printed antennas are arranged between the metal conductors off-center with respect to the central axis of the transmission line.

18. The instrument as claimed in claim 17, wherein the RF detectors are grouped together in modules, each of the modules comprising a string of eight RF detectors and one microcontroller.

19. The instrument as claimed in claim 2, wherein the RF detectors are grouped together in modules, each of the modules comprising a string of eight RF detectors and one microcontroller.

20. The instrument as claimed in claim 3, wherein the RF detectors are grouped together in modules, each of the modules comprising a string of eight RF detectors and one microcontroller.
